(19) United States
(12) Patent Application Publication
(10) Pub. No.: US 2021/0378499 A1
(43) Pub. Date: Dec. 9, 2021

(54) LARYNGOSCOPE

(71) Applicant: Children's Hospital Medical Center of Akron, Akron, OH (US)

(72) Inventor: Anton Milo, Akron, OH (US)

(73) Assignee: Children's Hospital Medical Center of Akron, Akron, OH (US)

(21) Appl. No.: 17/340,726

(22) Filed: Jun. 7, 2021

Related U.S. Application Data
(60) Provisional application No. 63/035,312, filed on Jun. 5, 2020.

(51) Int. Cl. A61B 1/267; A61B 1/00; A61B 1/06

(52) U.S. Cl. CPC A61B 1/2673; A61B 1/00066; A61B 1/0684; A61B 1/00103

(57) ABSTRACT

A laryngoscope may include a handle and a blade. The handle and blade may be a single piece and the handle may include undulating contours configured to ergonomically fit the user's hand. The blade may have a first end proximate the handle and a second end oppositely disposed from the first end. The blade end may include an anatomically contoured or undulating profile at the second end configured to correspond with an associate epiglottis and tongue of a patient. The laryngoscope may be disposable and configured for a single use. The blade may have an unilluminated state and an illuminated state.

### CROSS REFERENCE TO RELATED APPLICATIONS

This application claims priority to provisional application having U.S. Ser. No. 63/035,312, entitled LARYNGOSCOPE, filed Jun. 5, 2020, which is incorporated herein by reference.

### BACKGROUND

A laryngoscope is a medical device utilized to view a person's larynx or trachea. A typical laryngoscope comprises a handle and a blade and is used to visualize an area of interest proximate the trachea. A laryngoscope is often used for tracheal intubation in which the laryngoscope enables a clinician to see the path of an endotracheal intubation tube as it passes under the epiglottis and through the vocal cords towards the trachea. Laryngoscopes can be categorized into either direct or indirect laryngoscopes. Direct laryngoscopes allow a clinician to view a patient's larynx or trachea via direct line of sight. In contrast, indirect laryngoscopes use a form of video technology such as a video camera or fiber optics to view the larynx or trachea.

### SUMMARY

This Summary is provided to introduce a selection of concepts in a simplified form that are further described below in the Detailed Description. This Summary is not intended to identify key factors or essential features of the claimed subject matter, nor is it intended to be used to limit the scope of the claimed subject matter.

A laryngoscope may comprise a handle comprising a first end and a second end. A blade may comprise a blade operably connected to the second end of the handle. The blade may further comprise a first end and a second end oppositely disposed from the first end. The laryngoscope may further comprise an anatomic undulating profile configured to substantially correspond with portions of an associated epiglottis and tongue of an associated patient.

To the accomplishment of the foregoing and related ends, the following description and annexed drawings set forth certain illustrative aspects and implementations. These are indicative of but a few of the various ways in which one or more aspects may be employed. Other aspects, advantages and novel features of the disclosure will become apparent from the following detailed description when considered in conjunction with the annexed drawings.

### BRIEF DESCRIPTION OF THE DRAWINGS

What is disclosed herein may take physical form in certain parts and arrangement of parts, and will be described in detail in this specification and illustrated in the accompanying drawings which form a part hereof and wherein:

FIG. 1 is a view of an exemplary laryngoscope in an environment of use according to various aspects described herein.

FIG. 2 is a perspective view of an exemplary laryngoscope according to various aspects described herein.

FIG. 3 is a side perspective view of the exemplary laryngoscope of FIG. 2.

FIG. 4 is a rear perspective view of the exemplary laryngoscope of FIG. 2.

FIG. 5 is another perspective view of the laryngoscope shown in FIG. 2.

FIG. 6 is a side perspective view of another exemplary laryngoscope according to various aspects described herein.

FIG. 7 is a side perspective view of another exemplary laryngoscope according to various aspects described herein.

FIG. 8 is a close up perspective view of an exemplary blade of a laryngoscope according to various aspects described herein.

FIG. 9 is another perspective view of the exemplary blade of FIG. 8.

FIG. 10 is another perspective view of the exemplary blade of FIG. 8.

FIG. 11 is another perspective view of the exemplary blade of FIG. 8.

FIG. 12 is a view of an anatomy of an exemplary patient according to various aspects described herein.

FIG. 13 is a view of an exemplary laryngoscope inserted into the anatomy of the exemplary patient of FIG. 12.

FIG. 14 is a view of an anatomy of an exemplary patient according to various aspects described herein.

FIG. 15 is a view of an exemplary laryngoscope inserted into the anatomy of the exemplary patient of FIG. 14.

FIG. 16 is a front perspective view of three exemplary laryngoscopes, where each laryngoscope uses a different exemplary blade design.

FIG. 17 is a rear perspective view of the three exemplary laryngoscopes of FIG. 12.

FIG. 18 is a top perspective view of the three exemplary laryngoscopes of FIG. 12.

FIG. 19 is a top perspective view of an exemplary laryngoscope according to various aspects described herein.

FIG. 20 is a bottom perspective view of the laryngoscope of FIG. 19.

FIG. 21 is a rear view of the laryngoscope of FIG. 19.

FIG. 22 is a front view of the laryngoscope of FIG. 19.

FIG. 23 is a left side view of the laryngoscope of FIG. 19.

FIG. 24 is a right side view of the laryngoscope of FIG. 19.

FIG. 25 is a top view of the laryngoscope of FIG. 19.

FIG. 26 is a bottom view of the laryngoscope of FIG. 19.

FIG. 27 is an exemplary implementation of a laryngoscope having an illuminating feature.

FIG. 28 is an exemplary implementation of a laryngoscope having another illuminating feature.

### DETAILED DESCRIPTION

The claimed subject matter is now described with reference to the drawings, wherein like reference numerals are generally used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, structures and devices are shown in block diagram form in order to facilitate describing the claimed subject matter.

While useful in their purpose, laryngoscopes may have some shortfalls. These shortfalls may include suboptimal sterilization techniques, inadequate blade design, insufficient illumination of the upper airway/larynx due to a small light path, and overall poor ergonomics that make handling of the laryngoscope difficult or uncomfortable. An improved laryngoscope may provide solutions to the shortfalls of other laryngoscope designs that are mentioned above.

In an example relating to sterilization techniques, the laryngoscope provided herein may comprise a one-piece design that is disposable for improved and simplified sterilization. In other examples, the blade may be removeably engaged from the handle for sterilization and configuration.

In another example, and in regard to blade design, the laryngoscope provided herein may comprise an anatomically contoured blade design. The anatomic contours of the blade may mitigate effort required to maneuver the tongue and to deflect the epiglottis into an appropriate position during a medical procedure. Current laryngoscopes may require particular maneuvering of the tongue and epiglottis using the blade of the laryngoscope. This can be difficult and often requires careful operation of the laryngoscope to ensure proper deflection of the tongue and epiglottis. The laryngoscope provided herein may mitigate these inefficiencies with regard to maneuvering the tongue or epiglottis. The improved laryngoscope may further comprise an overall curvature that can improve effectiveness of the laryngoscope or mitigate the applied force needed to view the larynx of a patient, for example.

In another example, with respect to the light path provided by the laryngoscope, the laryngoscope provided herein may provide sufficient light to an area of interest to ensure a clear path of insertion of the endotracheal intubation tube as it is positioned past the epiglottis and vocal cords towards the trachea. This may provide improvements compared to current laryngoscope designs as the light path can often be narrow and become obstructed with blood or tissue during a medical procedure. This can require the clinician to pause or stop the medical procedure to clean the laryngoscope. The laryngoscope provided herein may mitigate these inefficiencies with regard to light production and visibility.

In another example, with respect to poor ergonomics of current laryngoscope design, the laryngoscope provided herein may comprise an anatomically contoured handle that may promote ergonomic functionality and improved control of the laryngoscope for safe and optimal visualization of a patient's airway.

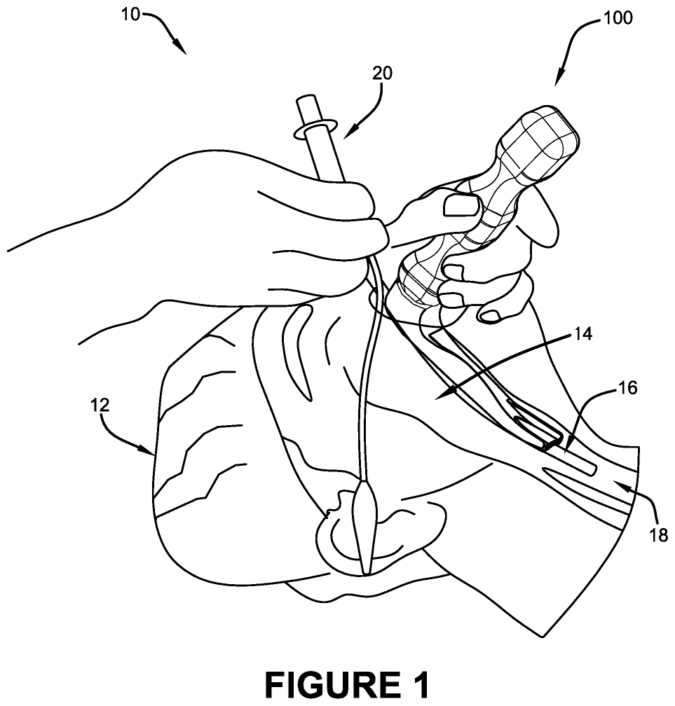

Referring now to FIG. 1, an exemplary environment 10 for utilizing an improved laryngoscope 100 is described. By way of example, a health care provider, such as a physician or other clinician may utilize the laryngoscope 100 for visualization or examination of a patient's 12 upper airway/larynx 14 by distraction of the upper airway structures. For instance, the laryngoscope 100 may be used to engage with and maneuver a patient's tongue and epiglottis for insertion of an endotracheal intubation tube 16 into the trachea 18 of the patient 12. At an end of the endotracheal intubation tube 16, a slip joint 20 may be selectably connected for airflow. With the endotracheal intubation tube 16 in place, a diagnostic procedure or surgical procedure may be performed. The laryngoscope 100 may also be used for airway management in anesthesia and for critical care practice as well as for other trauma scenarios. In other examples, the laryngoscope 100 may be utilized for diagnostic evaluation of the upper airway 14 and/or for therapeutic procedures.

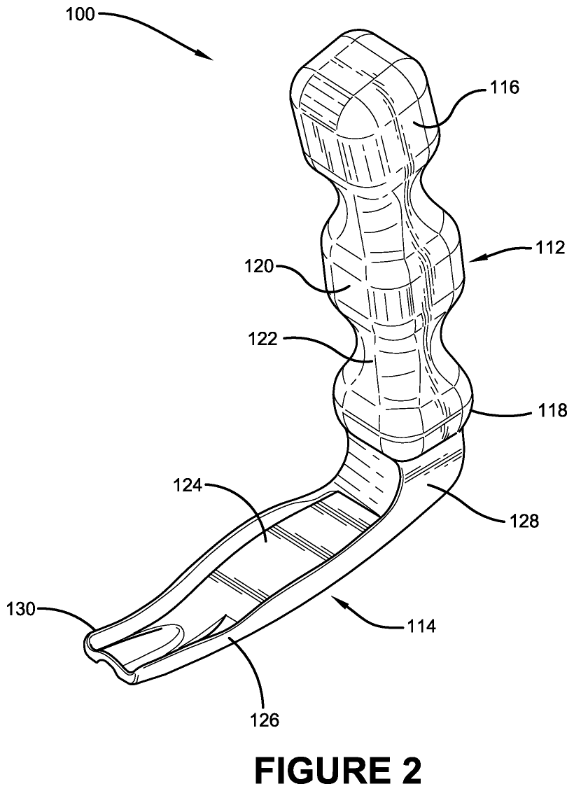

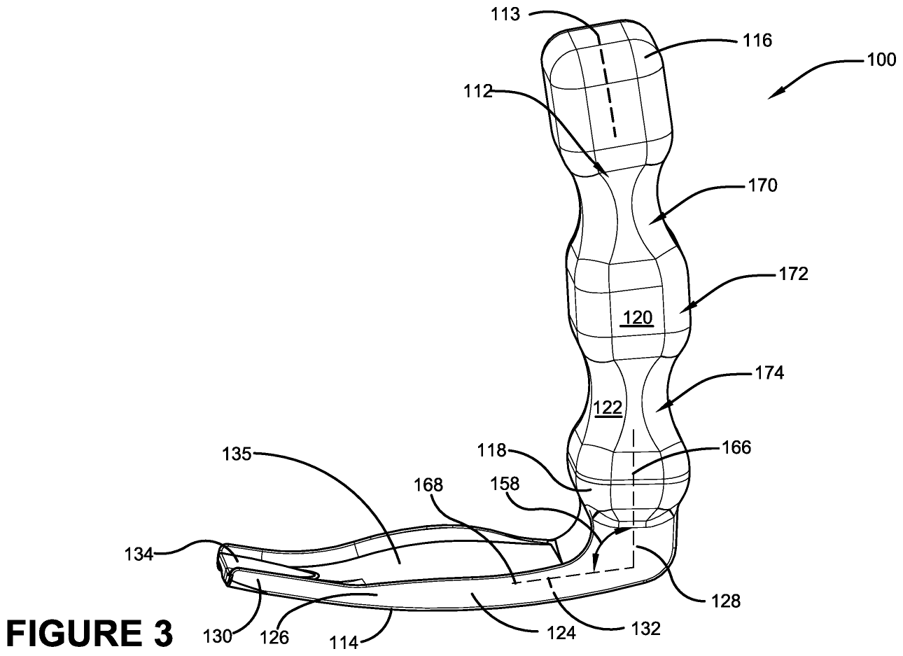

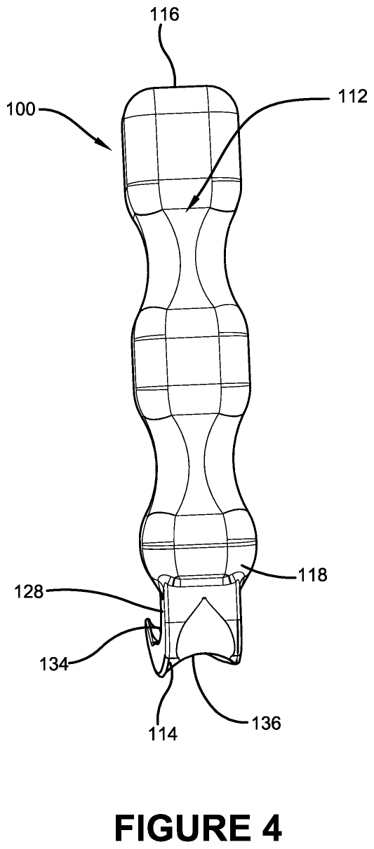

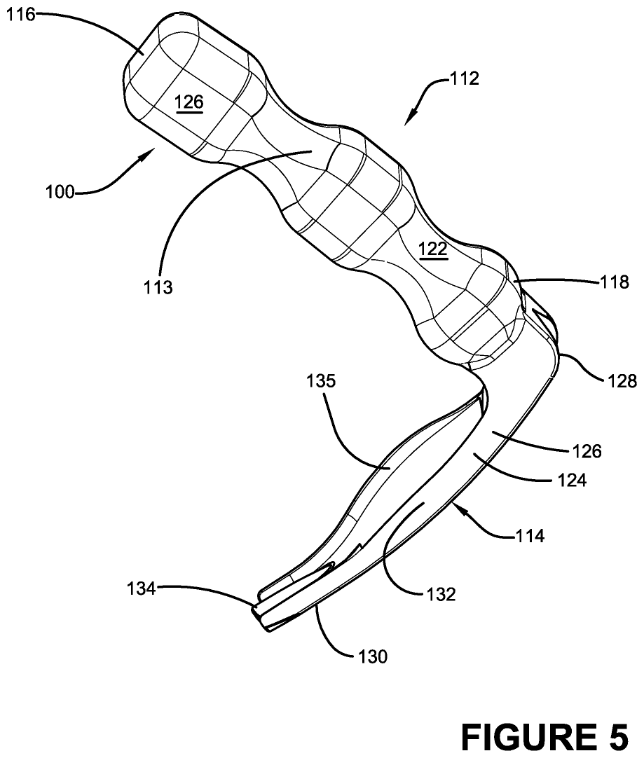

With reference to FIGS. 2-5, the laryngoscope 100 is shown. The laryngoscope 100 may comprise a handle 112 and a blade 114 operably connected to the handle 112. The handle 112 may comprise a first end 116 and a second end 118 oppositely disposed from the first end 116. The handle may comprise a body portion 120 extending from the first end 116 to the second end 118, where the body includes an outer surface 122. The blade 114 may comprise a first end 128 proximate the second end 118 of the handle 112 and a second end 130 oppositely disposed from the first end 128. The blade 114 may further comprise a body portion 124 extending from the first end 128 to the second end 130. The body portion 124 may comprise an outer surface 126.

In an exemplary implementation, the handle 112 may be operably connected to the blade 114. In another implementation, the handle 112 may be fixedly engaged with the blade 114. In yet another implementation, the handle 112 may be fixedly engaged with the blade 114 at an angle 158 that may provide an improved functionality of the laryngoscope 100. In this implementation, the handle 112 may comprise an axis 166 extending from the first end 116 of the handle 112 to the second end 118 of the handle 112. The blade 114 may comprise an axis 168 extending from the first end 128 of the blade 114 to the second end 130 of the blade 114. The axis 168 of the blade 114 can be oriented at an angle 158 compared to the axis 166 of the handle 112. In an example, the axis 168 of the blade 114 may be oriented at about a 90-degree angle to the axis 166 of the handle 112. In another example, the angle 158 may be chosen to be between 85 degrees to 95 degrees. Yet, in other examples, the angle 158 may be chosen according to sound engineering judgment. For instance, the angle 158 may be chosen according to a number of physical characteristics (e.g., size, weight, age, etc.) of an associated patient.

By way of example, the angle 158 between the handle 112 and the blade 114 may provide benefits compared to current laryngoscope design. Clinicians may face difficulties when positioning a laryngoscope within a patient's mouth and airway 14. These difficulties may lead to the clinician or operator positioning conventional laryngoscopes in such a manner that the conventional laryngoscopes abut a portion of a patient's teeth. In some situations, conventional laryngoscopes may be (e.g., either intentionally or unintentionally) operated as a lever to elevate the jaw or tongue of the patient using the teeth of the patient as a fulcrum point. This may lead to undesired positioning. The laryngoscope 100 may be configured such that the angle 158 between the handle 112 and the blade 114 mitigates unwanted contact with the patient's teeth or other sensitive internal organs. In this manner, a clinician may elevate the jaw or tongue of the patient 12 with minimal force and minimal contact with the patient's teeth to view the larynx or airway 14 of the patient. Those skilled in the art will appreciated the numerous benefits associated with the improved laryngoscope design described herein.

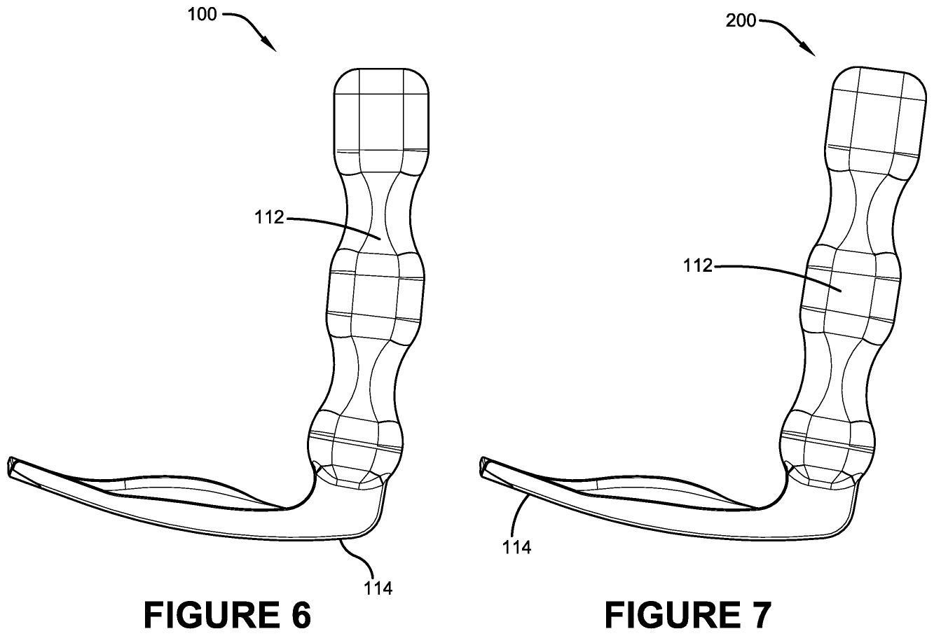

In an implementation illustrated in FIGS. 6 and 7, the handle 112 may be straight, tapered, or curved to conform to the user's hand. For instance, the handle 112 may be of differing lengths, widths, or shapes as appropriate for physical characteristics (e.g., size, weight, age, etc.) of the patient 12. FIG. 6 illustrates the laryngoscope 100 comprising a handle 112 that is curved. FIG. 7 illustrates another exemplary implementation of a laryngoscope 200. Laryngoscope 200 may be similar in all aspects compared to laryngoscope 100 except that laryngoscope 200 may comprise a handle 212 that is substantially straight and not curved.

In one non-limiting example, the handle 112 of laryngoscope 100 may be about 15 cm in length between the first end 116 and the second end 118. In other examples, the handle 112 may be modified to fit an associated hand of a user of the laryngoscope 100. It should be appreciated that the size and physical characteristics of patients and/or users of the laryngoscope 100 may vary and that physical dimensions of the laryngoscope 100 may be adjusted according to sound engineering judgment and physical requirements. Likewise, similar adjustments and configurations may be made for other implementations, such as with laryngoscope 200.

With reference to FIGS. 2-7, the handle 112 may comprise a plurality of undulating curves to conform to an associated user's hand. In an implementation, the handle 112 comprises a series of undulating curves 170, 172, 174. For example, the undulating curves 170, 172, 174 may comprise three higher sections and two lower valley type sections. The higher section have a larger diameter than the lower valley type sections. The undulating curves 170, 172, 174 may provide an ergonomic location in which the user may place his or her hand to grip around the handle 112 of the laryngoscope 100. In an implementation, the body portion 120 of the handle 112 may further comprise a coating of rubber, elastomeric, or any other suitable polymeric material to enable the handle 112 to be gripped by the associated user. For example, the coating may improve grip or friction between the handle 112 and a hand of the associated user. The coating may also improve the ergonomics or comfort associated with the handle.

In one non-limiting example, the handle 112 may have a partial or full cavity 113 contained therein. In another non-limiting example, the blade 114 may be partially hollow or completely hollow to define a cavity 132. In one implementation, the blade cavity 132 may be in communication with the handle cavity 113 to form a passageway. In this implementation, the handle 112 and the blade 114 of the laryngoscope may be a one-piece and substantially hollow design. This may reduce weight and/or manufacturing costs associated with the laryngoscope 100, for example.

In certain implementations, the handle 112 may be fixedly engaged to the blade 114. In other implementations, the handle 112 may be removably engaged to the blade 114. In these implementations, the blade 114 can be removed from the handle 112 for cleaning, disposal, etc. One skilled in the art will appreciate the benefits of a disposable blade 112. For example, benefits may include ease of sanitation and configurability of blade sizes and designs.

In other implementations, the handle 112 and the blade 114 may be manufactured as a one-piece design. Benefits of a one-piece laryngoscope design may include ease of sanitation. In an example, sanitation of the laryngoscope is not required. Rather, the laryngoscope is configured for one use and may be disposed of after a procedure.

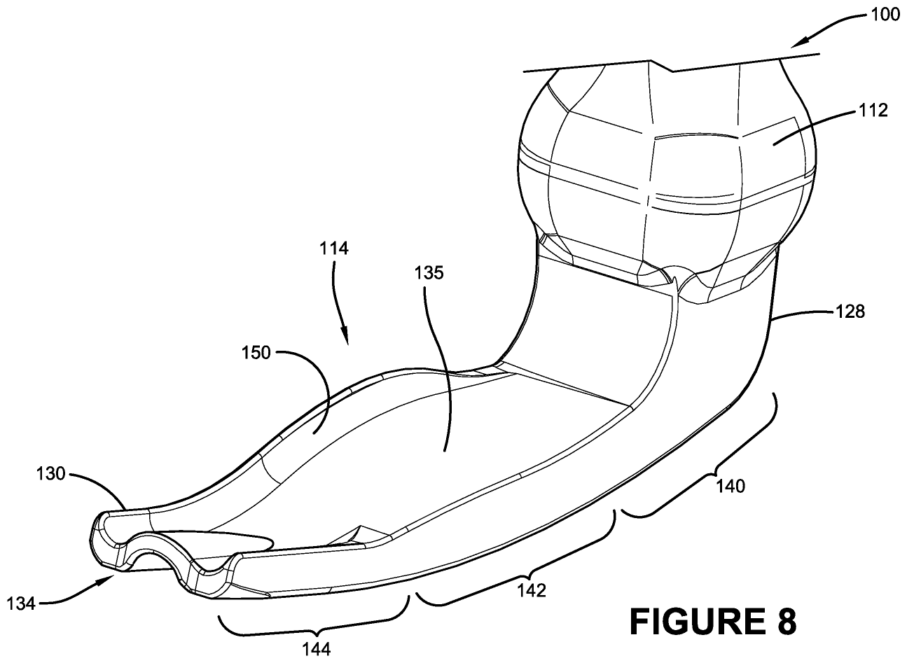

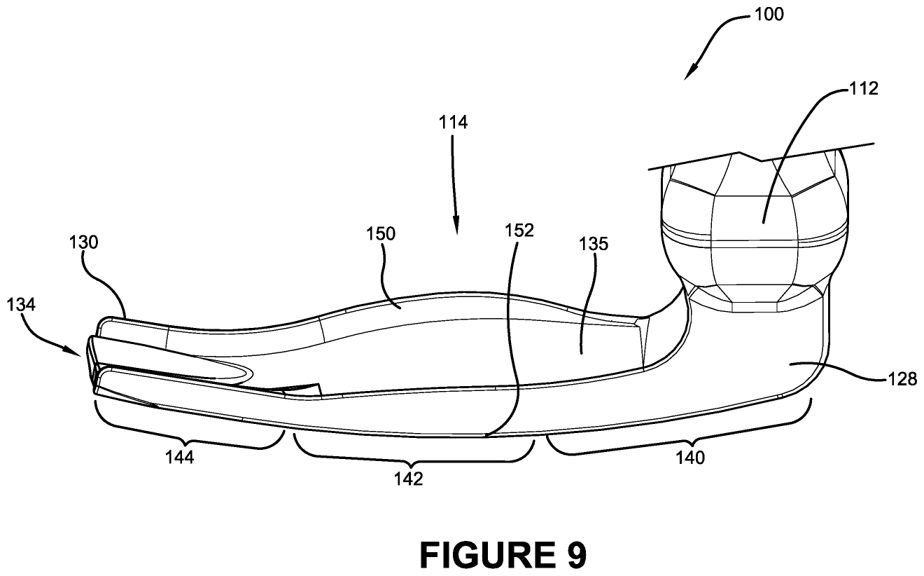

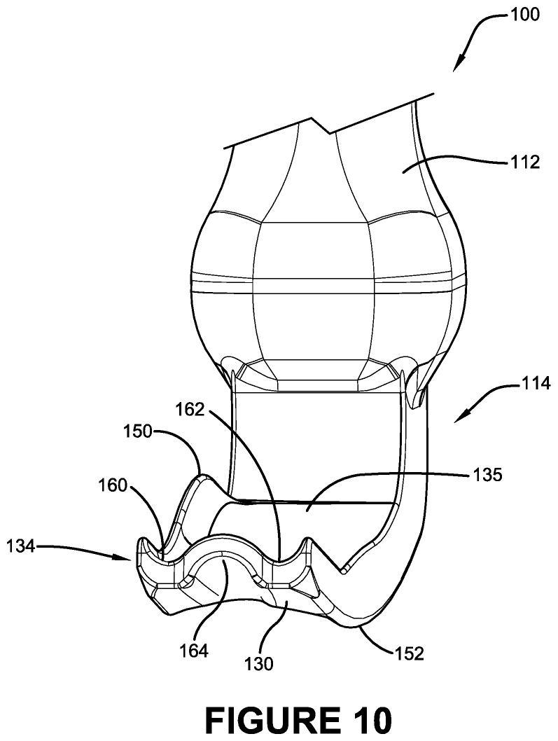

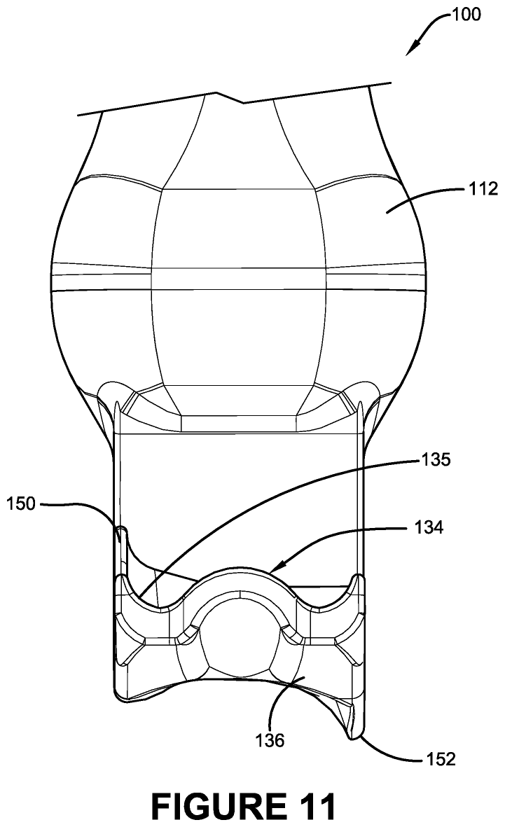

With reference to FIGS. 8-11, the blade 114 may comprise a plurality of anatomically contoured segments. In an implementation, the blade 114 comprises a first segment 140, referred to as a proximal segment, a second segment 142, referred to as a middle segment, and a third segment 144, referred to as a distal segment. The first segment 140 may be located proximate the first end 128 of the blade 114 closest to the handle 112. The third segment 144 may be located proximate the second end 130 of the blade 114. The second segment 142 may be located between the first segment 140 and the third segment 144. Further, the blade 114 may comprise a superior/lingual surface 135 and an inferior (airway 14) surface 136. By way of example, the superior surface 135 may engage the tongue of a patient. The inferior surface 136 may be directed towards the airway 14 of the patient 12.

In an implementation, the first segment 140 may be substantially flat on both the superior surface 135 and the inferior surface 136. The substantially flat configuration of the first segment 140 may mitigate harmful contact with a patient's teeth (e.g., dentition). As described above, contact with a patient's teeth by a laryngoscope during a medical procedure may be undesirable. In some circumstances, slight contact with dentition is unavoidable or may occur accidentally. The substantially flat superior surface 135 and inferior surface 136 of the first segment 140 may help to mitigate harmful contact with the patient's dentition by providing a safe and innocuous surface if contact with dentition is unavoidable.

The second segment 142 may comprise at least one anatomically contoured element, or and in some implementations, may comprise a plurality of anatomically contoured elements. In an implementation, the second segment 142 may comprise a first anatomically contoured element 150 located on the superior surface 135 of the blade 114. The first anatomically contoured element 150 may engage with and be used to displace (e.g., sweep) the tongue of the patient 12 to the left of the patient's mouth. The second segment 142 may further comprise a second anatomically contoured element 152 located on the inferior surface 136 to further engage the tongue of the patient 12 or to engage any other surface. For example, in some non-limiting implementations, the second anatomically contoured element 152 may engage the endotracheal intubation tube 16 as it is maneuvered into the trachea 18 of the patient 12.

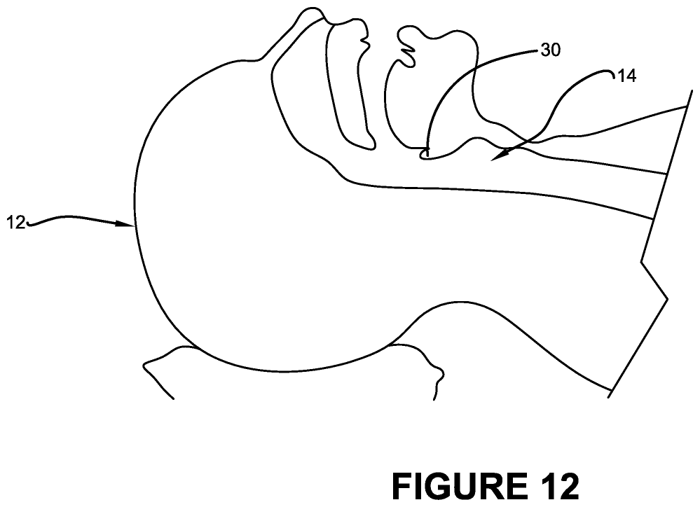

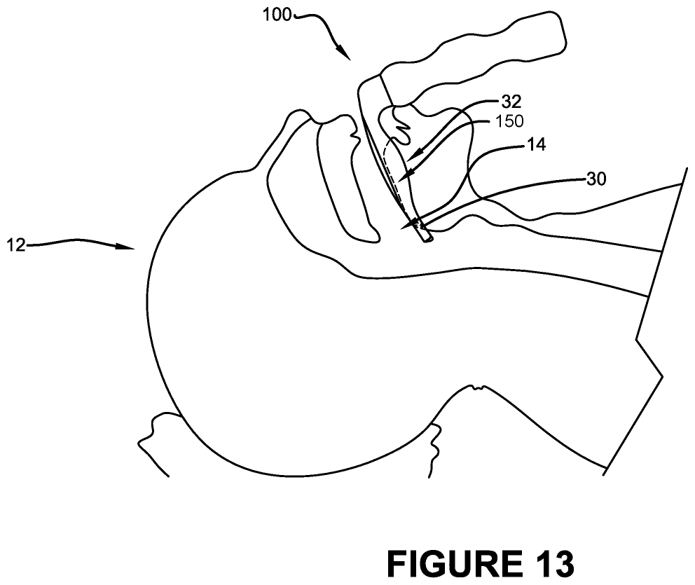

In an exemplary implementation, the first anatomically contoured element 150 may be opposite of the second anatomically contoured element 152 such that the first and the second anatomically contoured elements 150, 152 form an asymmetric (e.g., twisted, canted, etc.) configuration proximate the second segment 142 of the blade 114. As illustrated in FIGS. 12 and 13, the asymmetric design of the first and the second anatomically contoured elements 150, 152 may mitigate effort required to maneuver or rotate the tongue 32 to the left side of the patient's 12 mouth (e.g., lingual mandible) by providing support for, or by acting as a guide. For example, a clinician may engage the tongue 32 with at least one of the anatomically contoured elements 150, 152 to maneuver or rotate the tongue to a side of a patient's 12 mouth. It should be appreciated that the laryngoscope 100 may comprise a first anatomically contoured element 150, a second anatomically contoured element 152, or any other additional anatomically contoured elements according to sound professional judgment or design constraints. It should also be appreciated that although the present design illustrates a configuration to displace the tongue to the left of the patient's mouth, the design could be altered to displace the tongue to the right of the patient's mouth without deviated from the scope of the invention.

In an exemplary implementation illustrated in FIGS. 12 and 13, a patient 12 has an epiglottis 30 and a tongue 32. In a procedure, a clinician may insert the blade 114 of the laryngoscope 100 into the patient's airway 14 proximate the right side of the patient's mouth. In this manner, the blade 114, and the first anatomically contoured element 152, may engage with the tongue 32 to displace the tongue 32 to the left side of the patient's mouth. Clinicians experienced with laryngoscopy procedures may be familiar with shortcomings of current blade designs. For example, there may be a tendency for the tongue to slip over current blades and to return to the tongue's natural position in the center of the mouth. In some examples, when the tongue 32 slips around current blades, laryngoscopes may engage with a patient's teeth, which can cause damage. The first anatomically contoured element 150 of the exemplary laryngoscope 100 may mitigate unwanted movement of the tongue 32 by capturing the tongue within the contours of the first anatomically contoured element 150.

With continued reference to FIGS. 8-11, the third segment 144 may comprise at least one anatomic contour, or in some implementations, a plurality of anatomic contours for improved functionality. In an exemplary implementation, the anatomic contour of the third segment 144 may be in the form of an undulating curvature 134. The undulating curvature 134 may comprise a series of concave and convex portions. In one implementation, illustrated in FIGS. 8-11, the undulating curvature 134 may have a W-shaped profile.

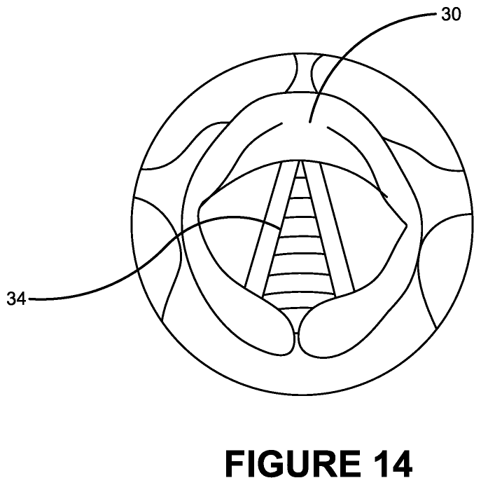

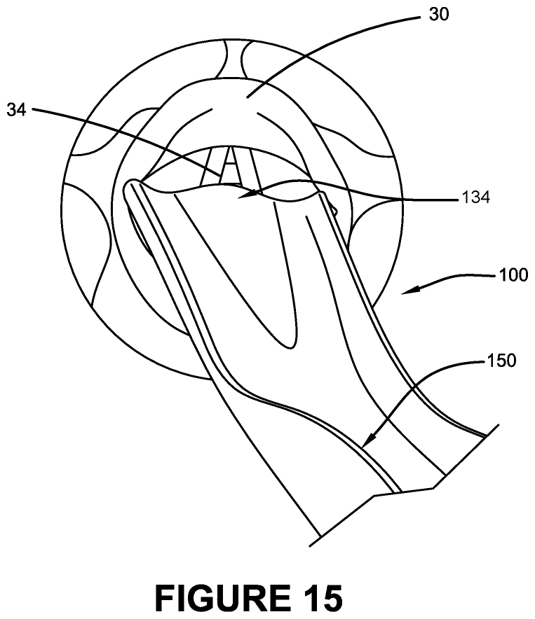

In an exemplary implementation illustrated in FIGS. 14 and 15, the undulating curvature 134 may be configured to lift and support different portions of the tongue 32 and/or epiglottis 30 to optimize visualization of the area of interest, such as the upper airway 14, larynx, or vocal cords 34. Further, in this regard, the W-shaped profile of the undulating curvature 134 may engage the epiglottis 30 to reveal the vocal cords 34 inferiorly. In addition, the superior surface 135 of the third segment 144 may be flat or may comprise an undulating curvature 134 comprising a series of concave and convex portions. The inferior surface 136 of the third segment 44 maybe flat, convex, or any shape chosen with sound engineering judgment to optimally facilitate complete visualization of the airway 14 and successful placement of an endotracheal tube 16 into the trachea 18 (intubation).

By way of illustration, the W-shaped anatomic contours or undulating curvature 134 can comprise two concave portions 160, 162, that may be similar in size and profile. The undulating curvature 134 may further comprise a convex portion 164 located between the concave portions 160, 162. It should be appreciated, however, that concave portions 160, 162 have opposite and corresponding convex portions and that convex portion 164 has an opposite and corresponding concave portion. For example, what appears as a convex portion from one perspective may be a concave portion for an opposite perspective.

In an example, the W-shaped configuration of the undulating curvature 134 may allow for better elevation and control over the epiglottis 30. The epiglottis 30 is a flap of tissue of varying shape and mass that covers the vocal cords and trachea 16. In an example, the tongue 32 must be maneuvered to the left side of a patient's mouth to allow for intubation or other required access to those areas. However, in most circumstances, the epiglottis 30 is difficult to control (e.g., may be slippery/floppy, etc.). With existing blades, which can be straight at the end, the epiglottis 30 can have a tendency to slip around the side edge of current blades and return (e.g., flop back) to its natural position covering the trachea 16. This requires a clinician to re-start the procedure of elevating the epiglottis 30 to allow for intubation, for example. This, in turn, may extend the duration of the intubation procedure, which is undesirable. The undulating curvature 134 of the laryngoscope 100 provided herein may provide additional capture or control over the epiglottis 30 which may mitigate unwanted movement of the epiglottis 30 during medical procedures.

In another example, the W-shaped configuration of the undulating curvature 134 may provide a guide for the endotracheal intubation tube 16 while being inserted into the trachea 16. In this example, the endotracheal intubation tube 16 may follow the undulating curvature 134 on the inferior surface 136 of the blade 114. One skilled in the art will appreciated that the undulating curvature 134 can guide the endotracheal intubation tube 16 into the trachea 16 of the patient while effectively holding the epiglottis 30 in an elevated/deflected position.

In an exemplary implementation, the blade 114 may gradually taper from the first end 128 towards the second end 130. In some implementations, the third segment of the blade 114 may be straight and not angled relative the body portion 124. Yet, in other implementations, the third segment 144 located proximate the second end 130 may be angled relative to the body portion 124. Alternatively, in other words the third segment 144 may be angled relative to the first segment 140 and the second segment 142. In this implementation, the first segment 140 and the second segment 142 may or may not be angled relative one another. By way of example, the angle of the third segment 144 relative the first and the second segment 140, 142 may allow the undulating curvature 134 to safely engage and elevate the epiglottis 30 with minimal effort as to view the airway 14 of the patient.

By way of example, a method of using the laryngoscope 100 can comprise a clinician inserting the laryngoscope 100 into the patient's 12 mouth. After inserting the laryngoscope 100, the undulating profile 134 of the blade 114 may substantially correspond with portions of the patient's epiglottis 30 and tongue 32. To continue the procedure, a clinician may maneuver the laryngoscope 100 within the associated patient's mouth to reposition the associated epiglottis 30 and tongue 32 of the patient 12 such that a medical or diagnostic procedure may be performed. For example, upon proper placement of the tongue 32 and elevation of the epiglottis 30, an endotracheal intubation tube 16 may be inserted into the trachea 18 of the patient 12.

It is also contemplated to be within the scope of the improved laryngoscope 100 may be compatible to be utilized simultaneously with various intubation tubes of varying diameters.

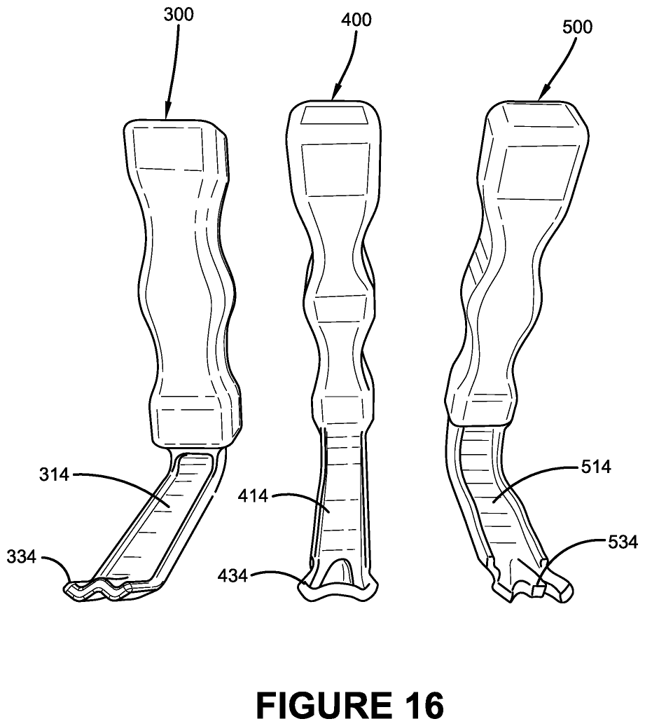

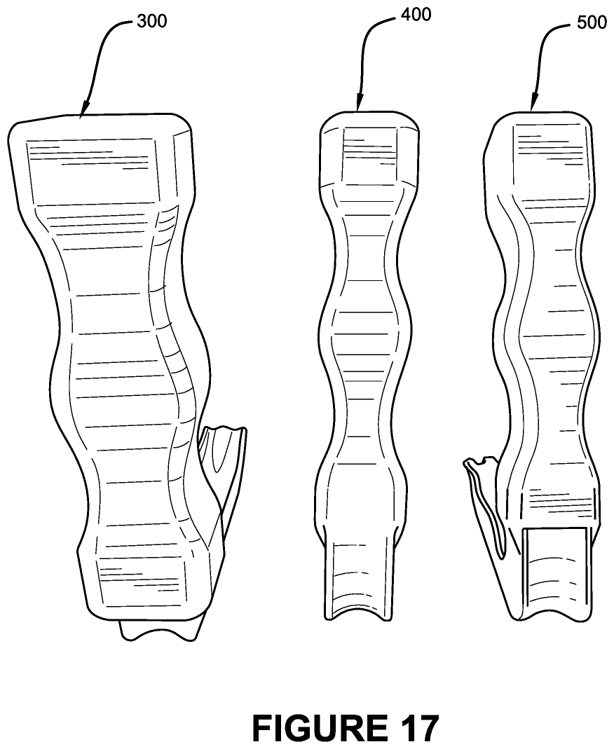

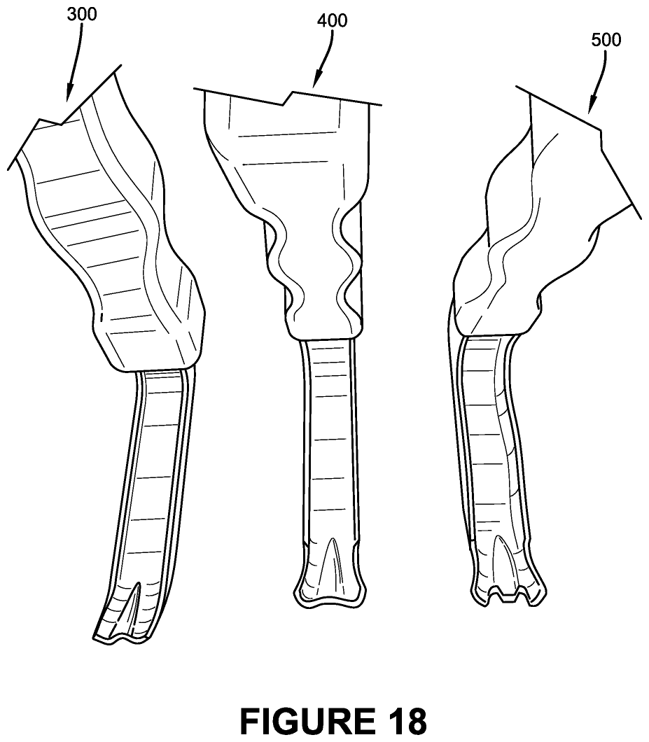

Turning to FIGS. 16-18, other exemplary implementations of a laryngoscope are shown. Laryngoscopes 300, 400, 500 may be similar in all aspects to laryngoscope 100 or 200 except as illustrated or described herein. For example, blade configuration for laryngoscopes 300, 400, 500 may differ according to sound engineering judgment. In an example, laryngoscope 300 may provide improved functionality during a laryngoscopy procedure by utilizing a W-shaped undulating profile 334 similar to that of undulating curvature 134 of laryngoscope 100. Laryngoscope 400 may provide improved functionality during a laryngoscopy procedure by utilizing a single contoured undulating profile 434. Laryngoscope 500 may provide improved functionality during a laryngoscopy procedure by utilizing a three-pronged anatomic profile 534.

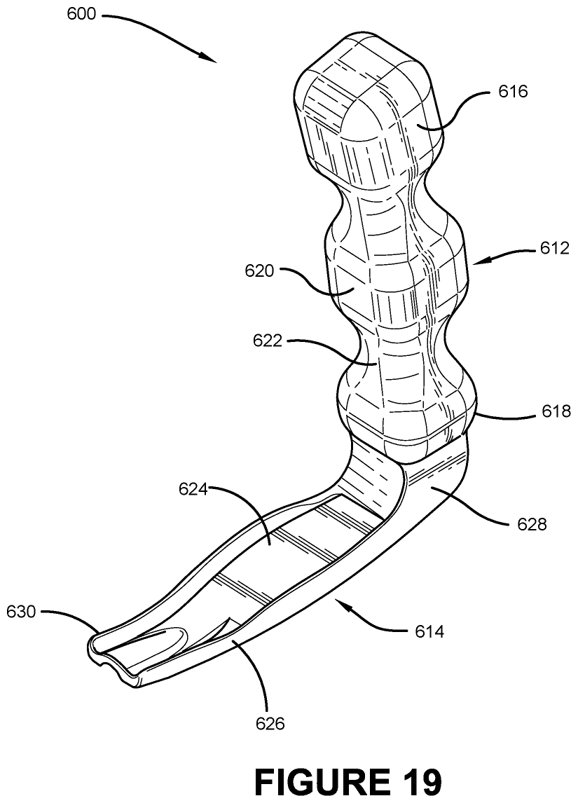

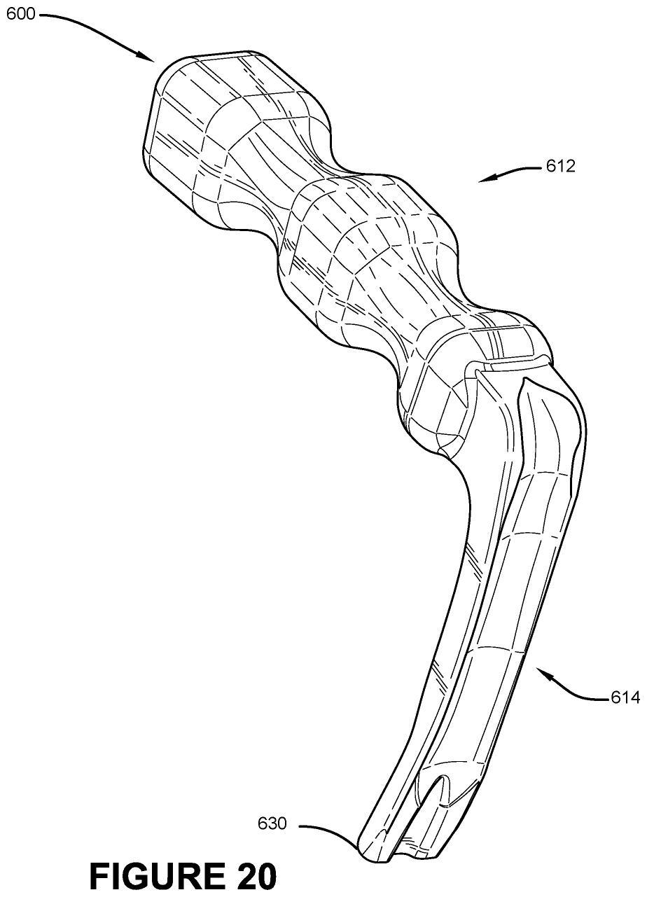

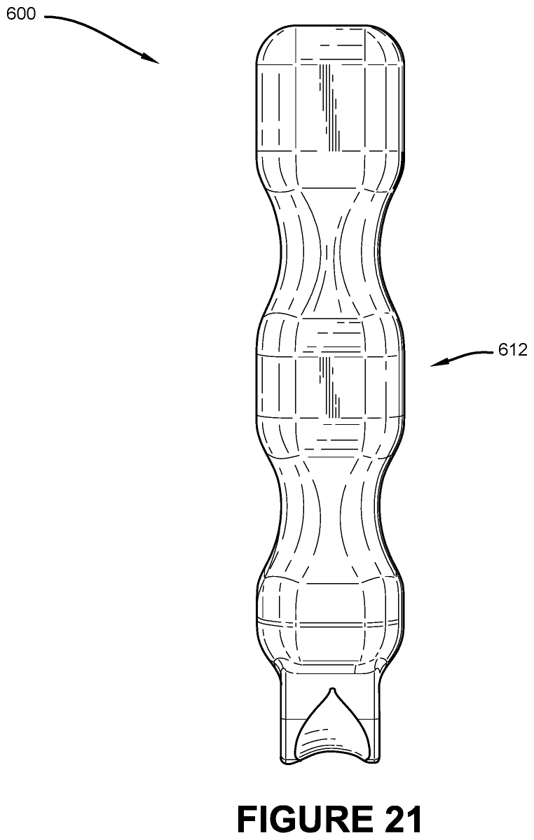

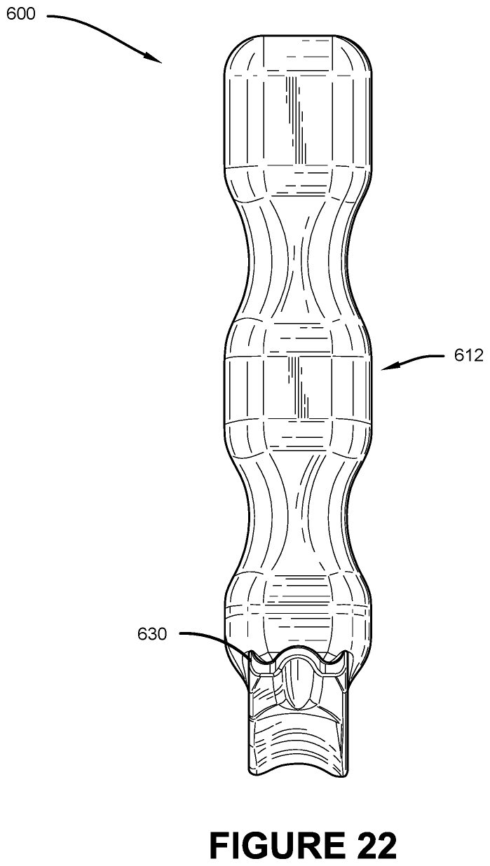

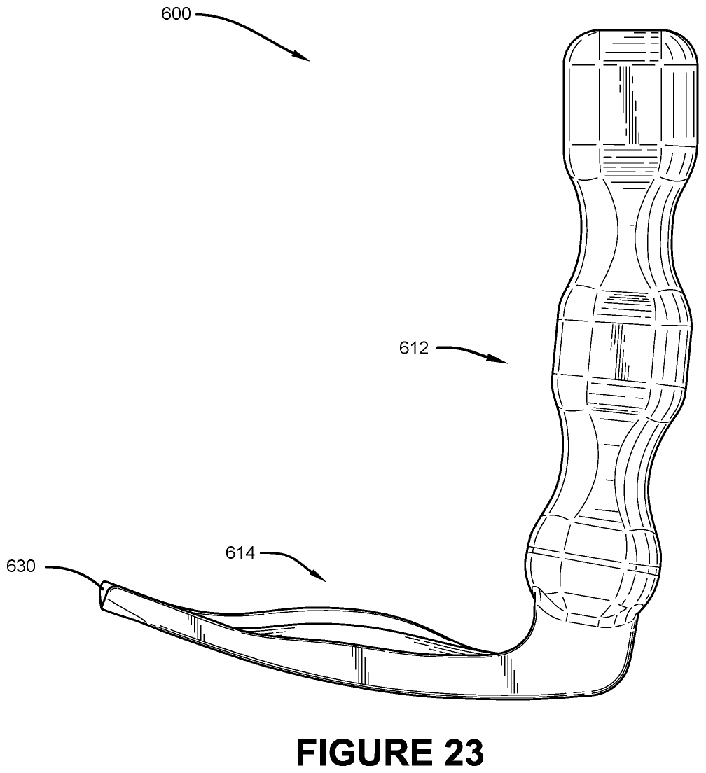

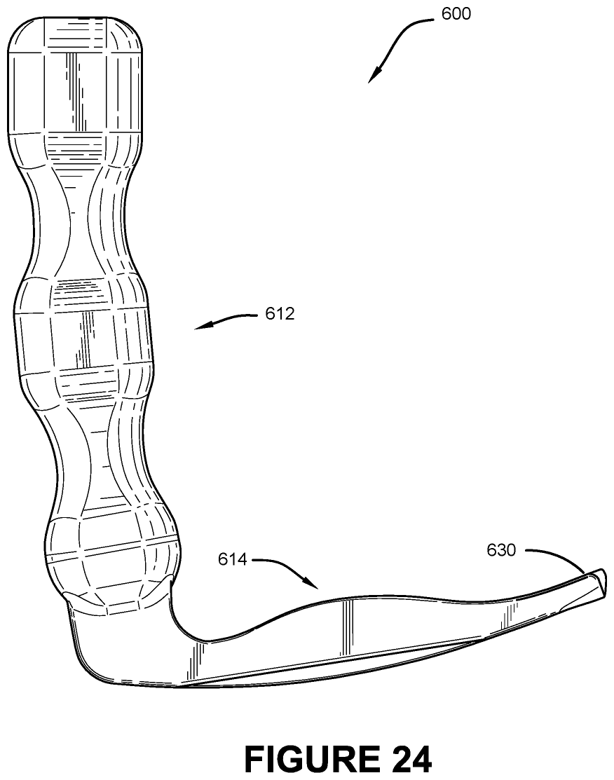

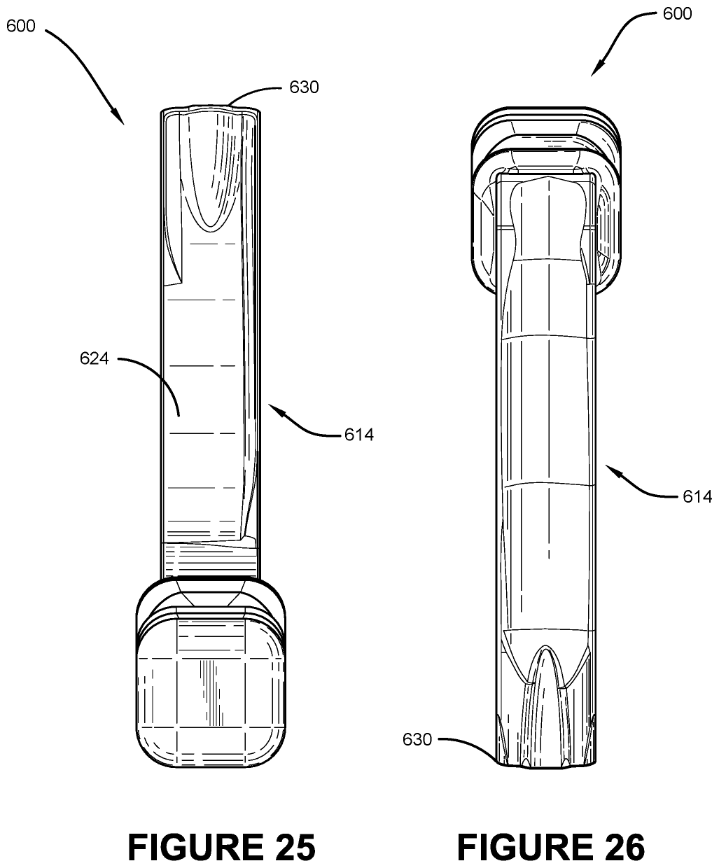

Turning to FIGS. 19-26, another exemplary implementation of a laryngoscope is shown generally at reference numeral 600. Laryngoscope 600 may be similar in all aspects to laryngoscopes 100 or 200 except as illustrated or described herein. Therefore like reference numerals make be used to identify like features in the drawings.

In an exemplary implementation, the blade 114 may have an illuminated state and an unilluminated state. To achieve illumination, the blade 114 may utilize a bulb or a LED, for example. Yet, in other implementations, the blade 114 can achieve an illuminated state without the use of a bulb (e.g., can be free of a bulb). Rather, the illumination may be achieved through a number of inventive ways. In one example implementation, illumination of the blade 114 may comprise a partial illumination of the blade 114 with discrete foci of light. In another implementation, the blade 114 may be completely illuminated with discrete foci of light. In another implementation, the blade may be translucent. The blade may be partially illuminated or fully illuminated. The translucent blade 114 may be configured to provide increased illumination up to complete illumination of an area of interest or an entire area of interest, such as the airway 14, for an intubated patient.

In yet another non-limiting example, the blade 114 may have gradual illumination of light from the first end 128 to the second end 130 having lesser illumination proximate the first end 128 and having greater illumination towards the second end 130. Gradual illumination may provide for reduced glare for the health care provider and improved functionality and visualization of the patient's 12 airway 14.

In one implementation to achieve blade illumination, the blade cavity 132 may be filled with a chemiluminescent fluid or material. In another example, the chemiluminescent fluid or material may be disposed in the handle cavity 113. By way of example, a single glass ampoule or other structure may be disposed in the handle 112 and/or the blade 114. The glass ampoule may be suspended in a second substance that when broken and mixed together, provide the chemiluminescent light. Any configuration that holds one material in a breakable containment structure to mix with the second substance may be utilized, such as a diaphragm or membrane. In another implementation, the structure may be punctured with a button (not shown) operably connected to the handle 112 or the blade 114. In another implementation, the structure may be punctured by shaking the laryngoscope 100. In another implementation, the spent chemiluminescent material may be removed from the handle cavity 113 and/or the blade 114 and a structure of unreacted chemiluminescent material is inserted therein for an additional use. In another implementation, light may be emitted from a luminescent coating disposed on the surface 126 of the blade 114. Examples of luminescent coatings may include without limitation, electroluminescent wires, tapes and panels. In yet another implementation, light may be transmitted within the blade 114 and emitted at appropriate points by way of light channeling or piping by acrylic, edge mirroring, or other light transmitting materials. In yet another implementation, light emitting diodes (LED) may be utilized as a light source at predetermined locations on the blade 114. Or, in other implementations, LEDS may be located proximate the handle 112 and light may be transferred (e.g., piped) via fiber optic to desired locations on the blade 114. It should be appreciated, however, that light may be transferred by other suitable means such as, but not limited to, acrylic light pipes, polycarbonate light pipes, silicon light pipes, mirrored devices, other by similar devices or methods according to sound engineering judgment.

In another implementation, the handle 112 may comprise a battery (not shown) disposed therein. It is contemplated that some implementations may not utilize batteries as a power source. In implementations utilizing batteries as a power source, the batteries can be in electrical communication with a light source that may be used to achieve the illuminated state of the blade 114. By way of example, a button or a switch (not shown) may be in electrical communication with the batteries. Pushing the button or switch may enable the batteries to power LEDs or other light source disposed in the blade 114. In this example, pushing the button or switch a second time can break the electrical circuit and turn the light off. In other implementations, the switch may be activated by twisting a portion of the laryngoscope 100 to an “ON” position and may stay activated until twisted to an “OFF” position. It should be appreciated that although the examples above provide for the use of a button or switch to control the supply of electricity to various electrical components, other forms of electrical devices may be used to control the supply of electricity. For example, devices such as throw switches, push buttons, toggle switches, rotary switches, potentiometers, transistors, MOSFETS, or similar devices may be used according to sound engineering judgment.

Light emission for the blade 114 may be of sufficient lumens or the equivalent of white light at illumination of the patient's 12 airway 14 for a sufficient amount of time for a health care provider to treat a patient. The time range for illumination may vary, and in one example may range from five minutes to about sixty minutes or longer. In another implementation, the illumination may have a duration from about ten minutes to about thirty minutes or longer. Shorter durations may be appropriate for diagnostic procedures, such as about ten minutes or longer. Longer durations may be appropriate for surgical procedures, such as about thirty minutes or longer.

In another implementation, the blade 114 may be integrated with the handle 114 such that the laryngoscope is a single unitary piece. For example, the blade 114 may be in an open position providing the health care provider ease of use since the laryngoscope 100 will be immediately positioned for use with an intended patient. A single piece laryngoscope 100 may also provide the additional benefit of more rapid illumination since the blade 114 is already in position. In other examples, the one-piece construction ensures no separation between the handle 112 and the blade 114. In other implementations, the laryngoscope 100 may be discarded after a single use, which alleviates the need for cleaning or sterilization.

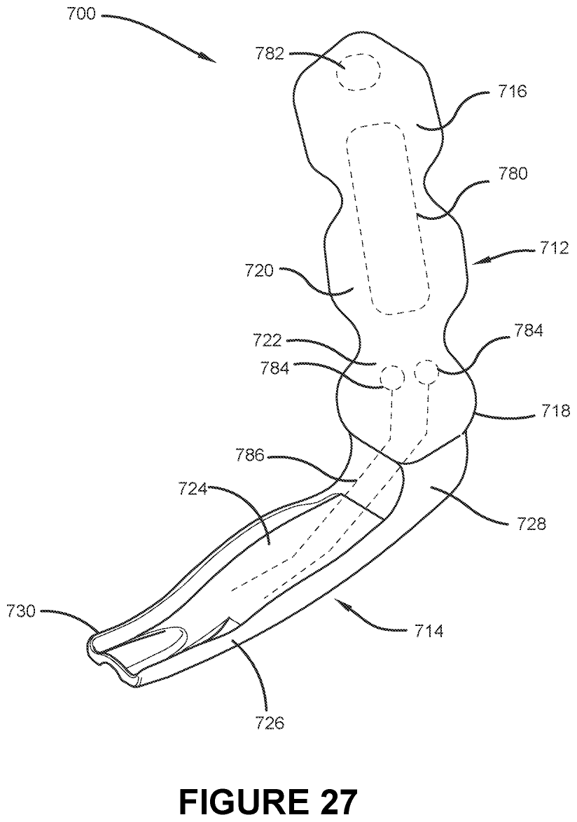

In an implementation illustrated in FIG. 27, an exemplary laryngoscope is shown generally at reference numeral 700. Laryngoscope 700 may be similar in all aspects to laryngoscopes 100 or 200 except as illustrated or described herein. Therefore like reference numerals make be used to identify like features in the drawings. For instance, laryngoscope 700 may comprise a battery 780 disposed within a cavity of the handle 712. The battery 780 may be in electrical communication with a button or switch 782. The button or switch 782 may activate a series of LEDs 784 located with the cavity of the handle 712. In this implementation, light emitted from the LEDs may be directed via fiber optic cables 786 to various locations on the blade 714. In other implementations, light emitted from the LEDs may be directed (e.g., piped) using various other means according to sound engineering judgment. It should be appreciated that the locations of the battery 780, the button 782, the LEDs 784, or the fiber optics 786 may vary according to sound engineering judgment. It should also be appreciated that the quantity of batteries 780, buttons 782, LEDs 784, or the fiber optics 786 may also vary according to sound engineering judgment.

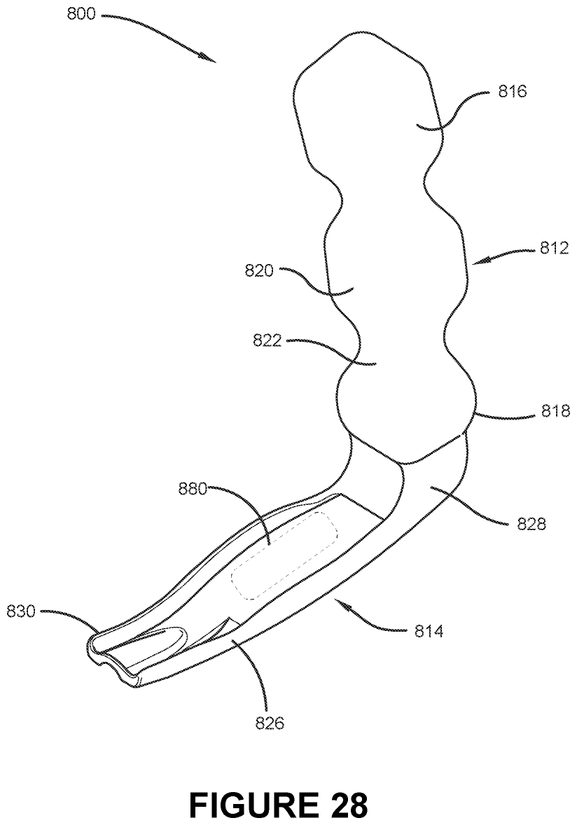

In an implementation illustrated in FIG. 28, an exemplary laryngoscope is shown generally at reference numeral 800. Laryngoscope 800 may be similar in all aspects to laryngoscopes 100 or 200 except as illustrated or described herein. Therefore like reference numerals make be used to identify like features in the drawings. For instance, as illustrated in FIG. 28, laryngoscope 800 may comprise a chemiluminescent fluid or material within a cavity of the blade 814. In another example, the chemiluminescent fluid or material may be disposed in the handle 812. By way of example, a single glass ampoule 880 or other structure may be disposed in the handle 812 and/or the blade 814. The glass ampoule 880 may be suspended in a second substance that when broken and mixed together, it can provide the chemiluminescent light. It should be appreciated, however, that any configuration that holds one material in a breakable containment structure to mix with the second substance may be utilized, such as a diaphragm or membrane.

The word “exemplary” is used herein to mean serving as an example, instance or illustration. Any aspect or design described herein as “exemplary” is not necessarily to be construed as advantageous over other aspects or designs. Rather, use of the word exemplary is intended to present concepts in a concrete fashion. As used in this application, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. Of course, those skilled in the art will recognize many modifications may be made to this configuration without departing from the scope or spirit of the claimed subject matter.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure which performs the function in the herein illustrated exemplary implementations of the disclosure. The laryngoscope 10 may be made of any medical grade material chosen with sound engineering judgment. Non-limiting examples may include plastic or polymeric material.

In addition, while a particular feature of the disclosure may have been disclosed with respect to only one of several implementations, such feature may be combined with one or more other features of the other implementations as may be desired and advantageous for any given or particular application. Furthermore, to the extent that the terms “includes,” “having,” “has,” “with,” or variants thereof are used in either the detailed description or the claims, such terms are intended to be inclusive in a manner similar to the term “comprising.”

The implementations have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

What is claimed is:

1. A laryngoscope, comprising:
a handle comprising a first end and a second end;
a blade operably connected to the second end of the handle, the blade further comprising:
a first end;
a second end oppositely disposed from the first end; and
an anatomic undulating profile configured to substantially correspond with portions of an associated epiglottis and tongue of an associated patient.

2. The laryngoscope of claim 1, wherein the blade further comprises a contoured side wall extending at least partially along a side of the blade in a direction from the first end of the blade to the second end of the blade, the contoured side wall configured to laterally displace and rotate portions of the associated epiglottis and tongue of the associated patient.

3. The laryngoscope of claim 1, wherein the blade further comprises a tip portion at the second end of the blade, the tip portion positioned at an angle respective to a body of the blade, and the tip portion comprising at least a portion of the anatomic undulating profile.

4. The laryngoscope of claim 1, wherein the handle is fixedly engaged with the blade, the handle comprising:
a plurality of undulating contours extending from the first end to the second end configured to ergonomically fit an associated user's hand.

5. The laryngoscope of claim 4, wherein the handle comprises a body extending from the first end of the handle to the second end of the handle, the body comprising a coating of one or more of a rubber or elastomeric material to configured to improve gripping properties of the handle.

6. The laryngoscope of claim 4, wherein the blade is selectably removable with the second end of the handle.

7. The laryngoscope of claim 6, wherein the blade is disposable and replaceable.

8. The laryngoscope of claim 4, wherein the blade and the handle are configured to be a single piece.

9. The laryngoscope of claim 4, wherein the handle comprises a cavity defined within the body of the handle.

10. The laryngoscope of claim 9, wherein the blade comprises a cavity defined therein, the cavity of the blade in communication with the cavity of the handle forming a passageway therebetween.

11. The laryngoscope of claim 4, wherein the blade comprises an axis extending from the first end of the blade to the second end of the blade, the handle comprises an axis extending from the first end of the handle to the second end of the handle, the axis of the blade oriented at an angle relative to the axis of the handle, the angle being between 85 degrees and 95 degrees.

12. The laryngoscope of claim 1, wherein the anatomic undulating profile is proximate the second end of the blade.

13. The laryngoscope of claim 12, wherein the anatomic undulating profile is a W-shape.

14. The laryngoscope of claim 1, wherein the blade has an illuminated state and an unilluminated state.

15. The laryngoscope of claim 14, wherein the illuminated comprises one or more of:
a light emitting diode;
a chemiluminescent fluid operably associated with an ampoule;
a luminescent coating; and
a battery.

16. A method of using a laryngoscope, the laryngoscope comprising a blade operably connected to a handle, the handle comprising a first end and a second end, the blade operably connected to the second end of the handle, the blade further comprising a first end, a second end oppositely disposed from the first end, and an anatomic undulating profile, the method comprising:
inserting the laryngoscope into an associated patient's mouth, the undulating profile of the blade substantially corresponding with portions of an associated epiglottis and tongue of the associated patient; and
maneuvering the laryngoscope within the associated patient's mouth to reposition the associated epiglottis and tongue of the associated patient such that a medical or diagnostic procedure may be performed.

17. The method of claim 14, wherein the blade further comprises a contoured side wall extending on a side of the blade from the first end of the blade to the second end of the blade, the contoured side wall configured to elevate portions of the associated epiglottis and to laterally displace the tongue of the an associated patient.

18. The method of claim 14, wherein the blade further comprises a tip portion at the second end of the blade, the tip portion positioned at an angle respective to a body of the blade, and the tip portion comprising at least a portion of the anatomic undulating profile.

19. The method of claim 14, wherein the handle is fixedly engaged with the blade, the handle comprising:
a plurality of undulating contours extending from the first end to the second end configured to ergonomically fit an associated user's hand.

20. A laryngoscope, comprising:
a handle comprising a first end and a second end;
a blade operably connected to the second end of the handle, the blade further comprising:
a first end;
a second end oppositely disposed from the first end; and
an anatomic undulating profile proximate the second end configured to substantially correspond with portions of an associated epiglottis and tongue of an associated patient,
wherein the handle and blade being a single piece and configured to be disposable.
